(19) United States
(12) Patent Application Publication
(10) Pub. No.: US 2001/0043911 A1
(43) Pub. Date: Nov. 22, 2001

(54) Deodorant composition

(75) Inventors: Toyoki Hagura, Sumida-ku (JP); Takayoshi Kajino, Sumida-ku (JP); Tetsuro Kamiya, Sumida-ku (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: KAO CORPORATION, Chuo-ku (JP)

(21) Appl. No.: 09/819,853

(22) Filed: Mar. 29, 2001

(30) Foreign Application Priority Data
Mar. 31, 2000 (JP) 2000-096967
Apr. 21, 2000 (JP) 2000-121005
Aug. 30, 2000 (JP) 2000-260116

(51) Int. Cl. A61K 7/32

(52) U.S. Cl. 424/65

(57) ABSTRACT

The present invention provides a deodorant composition which exhibits excellent and long-lasting deodorant effect against body odor, dries quickly on the skin, imparts soft feel to the skin, provides pleasant dry touch to the skin, and can be handled with ease. The deodorant composition contains a disinfectant or an antiperspirant and a thickener. The composition dries within a drying time of 30 seconds, has a dynamic friction resistance of 50% or less and a viscosity of 1,000-30,000 mPa·s.

### TECHNICAL FIELD

The present invention relates to a deodorant composition which has enhanced deodorant properties, dries quickly, and imparts pleasant smooth feeling to the skin, thereby attaining excellent soft and dry touch to the skin.

### BACKGROUND ART

Smell of sweat, underarm odor, foot odor, and other body odor are generated when amino acids, neutral fat, fatty acids, cholesterol, or similar substances contained in sweat secreted from eccrine glands and apocrine glands, are decomposed into odoriferous substances by bacteria on the skin. A number of methods have been undertaken so as to suppress body odor, including a method employing antiperspirants exhibiting dermal protein astringent action, a method employing a disinfectant (or a germicide) so as to prevent proliferation of bacteria on the skin, a method of converting lower fatty acids, which are considered to cause underarm odor or foot odor, into metal salts thereof, and a masking method using a perfume or eau de cologne.

However, active ingredients for preventing such odor are easily lost due to perspiration and physical friction, and therefore, odor-preventive effects do not last for long periods of time. To solve this problem, several techniques have been proposed, which include use of a linear polymer in an attempt to maintain effects of disinfectants and antiperspirants for a long time by preventing removal of active ingredients (Japanese Patent Application Laid-Open (kokai) No. 62-108809); and incorporation of disinfectants in large amounts. However, the method making use of a linear polymer causes a bad feeling upon application of the deodorant product, whereas use of large amounts of disinfectants causes a bad touch to the skin.

Moreover, Japanese Patent Application Laid-Open (kokai) No. 61-155318 discloses a liquid antiperspirant/deodorant cosmetic composition containing a lower alcohol and a higher branched alcohol. Furthermore, Japanese Patent Application Laid-Open (kokai) No. 4-211008 discloses an antiperspirant composition containing a lower alcohol and clay which has undergone hydrophobic treatment. These compositions exhibit insufficient deodorant effect and dry touch to the skin.

Deodorant compositions are typically formulated as liquids, sprays, roll-on products, aerosols, solids, powders, etc. Liquid forms may be superior to spray forms, in that the intended amount of the product can be applied without wasting. However, since in the summertime the skin tends to be moistened, drying of a liquid deodorant product applied to the skin takes time, and thus refreshing feeling cannot be obtained instantaneously. Moreover, liquid deodorant products are problematic in that they may spill from the palms, smudging the floor and clothes.

In view of the foregoing, an object of the present invention is to provide a deodorant composition which has enhanced deodorant properties, which provides soft and dry touch to the skin, and which can be handled with ease.

### DISCLOSURE OF THE INVENTION

The present inventors have found that reduction in dynamic friction resistance (which will be defined herein later) between the deodorant composition and the skin, speed-up of drying of the composition, and regulation of the viscosity of the composition to fall within a specific range provides an excellent deodorant composition which can be conveniently used and is capable of imparting pleasant, dry feeling to the skin instantaneously upon application to the skin even when the skin is in a moistened state.

Accordingly, the present invention provides a deodorant composition containing a disinfectant or an antiperspirant, and a thickener, which composition dries within a drying time of 30 seconds, exhibits a relative dynamic friction resistance of 50% or less, and has a viscosity of 1,000-30,000 mPa·s.

The present invention also provides a deodorant method comprising applying to the skin a composition containing a disinfectant or an antiperspirant, and a thickener, which composition dries within a drying time of 30 seconds, exhibits a relative dynamic friction resistance of 50% or less, and has a viscosity of 1,000-30,000 mPa·s.

### BEST MODES FOR CARRYING OUT THE INVENTION

The disinfectant or antiperspirant to be incorporated into the deodorant composition of the present invention may be used in combination. When one is used solely, a disinfectant (or a germicide) is preferably used. Examples of the disinfectant include nonionic disinfectants such as 3,4,4-trichlorocarbanilide (TCC), resorcin, phenol, hexachlorophene, triclosan, and isopropyl methyl phenol; cationic disinfectants such as benzalkonium chloride, benzetonium chloride, alkyl trimethyl ammonium chloride, and zinc pyrithione; and anionic disinfectants such as sorbic acid and salicylic acid. Examples of the antiperspirant include aluminum hydroxychloride, aluminum chloride, aluminum sulfate, basic aluminum bromide, aluminum phenolsulfonate, tannic acid, aluminum naphthalene sulfonate, basic aluminum iodide, zirconium salts, aluminum-zirconium complex salts, and zinc p-phenolsulfonate. The disinfectant is preferably contained in an amount of 0.01-5.0% by weight (hereinafter simply referred to as %), particularly preferably 0.05-3.0%, on the basis of the entirety of the composition. The antiperspirant is preferably contained in an amount of 0.1-30% by weight, particularly preferably 1.0-15.0%, on the basis of the entirety of the composition.

The deodorant composition of the present invention dries within a drying time of 30 seconds. The lower limit is generally 3 to 5 seconds. Preferably, the deodorant composition dries within a drying time of 25 seconds, and in this case also, the lower limit is generally 3 to 5 seconds. More preferably, the deodorant composition dries within a drying time of 20 seconds, and in this case also, the lower limit is generally 3 to 5 seconds.

In the present description, the drying time is defined as follows. First, an individual panel stays in a room of constant temperature and constant humidity (20° C., 60% RH) for 10 minutes. Subsequently, a deodorant composition (0.3 g) is applied uniformly over a certain area of the inner side of the tester's forearm (100 cm²; 5×20 cm), and, using the forefinger and middle finger, the tester rubs the composition-applied area in the lengthwise direction in 15 reciprocating movements in 10 seconds so as not to apply pressure to the skin. After a certain time, the tester acknowledges that “catchy” resistance is no longer felt. This time is defined as the “drying time.”

The deodorant composition of the present invention has a relative dynamic friction resistance of 50% or less, and the lower limit is generally 3% or 5%. Preferably, the relative dynamic friction resistance is 40% or less, and in this case also, the lower limit is generally 3 seconds or 5 seconds. More preferably, the relative dynamic friction resistance is 30% or less, and in this case also, the lower limit is generally 3 seconds or 5 seconds.

As used herein, the relative dynamic friction resistance refers to a parameter indicating actual feeling of the skin in terms of pleasant dry, smooth feel perceived after application of the deodorant composition to the skin, and is measured through the following method.

Measurement of Dynamic Friction Resistance

Surface of a mat (5 cm×12 cm) made of acrylonitrile butadiene rubber (hereinafter referred to as NBR) (for example, Kreseed (product of Kureha Elastomer K.K.; model MB265N; thickness=1 mm) which is specified in JIS K6380 may be used) is washed with ethanol, and thereafter, the mat is affixed onto a measurement table by use of a pressure-sensitive adhesive double coated tape. Independently, an ethanol-cleansed NBR mat (3 cm×3 cm) is set on the upper arm of a surface tester (manufactured by Shinto Kagaku K.K., model HEIDON-14D). With the application of a load of 200 g, the mat-bearing arm is moved against the surface of the NBR mat secured on the measurement table for a horizontal distance of 7 cm at a movement speed of 5 cm/sec, to thereby measure the dynamic friction resistance between the two mats (referred to as dynamic friction resistance A). The same measurement is performed after a deodorant composition (0.1 g) is applied uniformly onto the upper surface of the test mat and sufficiently dried, to thereby measure the dynamic friction resistance between the two mats to which the deodorant composition has been applied (referred to as dynamic friction resistance B). The relative dynamic friction resistance is calculated by the following equation:

Dynamic friction resistance (%)=(B/A)×100

Regarding the viscosity (25° C.) of the deodorant composition of the present invention, the upper limit is 30,000 mPa·s, preferably 20,000 mPa·s, more preferably 10,000 mPa·s, as measured by use of a B-type viscometer at 12 rpm for 60 sec. The lower limit is 1,000 mPa·s, preferably 1,500 mPa·s, more preferably 2,000 mPa·s. The viscosity preferably falls within the range of 1,000-30,000 mPa·s, preferably 1,500-20,000 mPa·s, more preferably 2,000-10,000 mPa·s. By adjusting the viscosity to fall within the above ranges, running-down of the composition which may occur during application can be prevented. The aforementioned viscosity of the deodorant composition can be attained by incorporating a thickener into the composition. Examples of the thickeners include (meth)acrylic polymers such as poly((meth)acrylic acid), cross-linked poly((meth)acrylic acid), (meth)acrylic acid-alkyl (meth)acrylate copolymers, and cross-linked (meth)acrylic acid-alkyl (meth)acrylate copolymers and cationic cross-linked copolymers. Other examples include hydroxyethyl cellulose, hydroxypropyl cellulose, carboxymethyl cellulose, xanthan gum, carrageenin, gelatin, methyl cellulose, ethyl cellulose, poly(vinyl alcohol), and derivatives thereof. Of these, poly((meth)acrylic acid), (meth)acrylic acid-alkyl (meth)acrylate copolymers, cross-linked poly((meth)acrylic acid), cross-linked (meth)acrylic acid-alkyl (meth)acrylate copolymers, and cationic cross-linked copolymers are preferred.

The cationic cross-linked copolymer is a substance having a cationic group in the molecule thereof and cross-linking structure. Examples include a cationic cross-linked copolymer (hereinafter referred to as copolymer (A)) which contains, as essential structural monomers, at least one cationic-group-containing vinyl monomer (hereinafter referred to as monomer (a₁)); at least one amido-group-containing vinyl monomer (hereinafter referred to as monomer (a₂)); and at least one cross-linkable vinyl monomer containing two or more vinyl groups in the molecule thereof (hereinafter referred to as monomer (a₃)).

Copolymer (A) may further contain, as a structural monomer, another vinyl monomer such as a (meth)acryloyl-group-containing vinyl monomer.

Examples of the monomer (a₁) which constitutes copolymer (A) include dialkylamino-group-containing (meth)acrylate esters or (meth)acrylamides represented by formula (1):

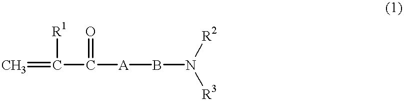

(wherein R¹ represents a hydrogen atom or a methyl group; each of R² and R³, which may be identical to or different from each other, represents a hydrogen atom or a C1-C4 linear or branched alkyl or alkenyl group; A represents an oxygen atom or an —NH— group; and B represents a C1-C4 linear or branched alkylene group) and acid-neutralized products, quaternary ammonium salts, and diallyl-type quaternary ammonium salts of amino-group-containing monomers such as dialkylamino-group-containing styrenes, vinylpyridines, and N-vinylheterocyclic compounds.

Examples of the aforementioned acids include hydrochloric acid, sulfuric acid, acetic acid, citric acid, succinic acid, adipic acid, and sulfamic acid. Examples of preferred quaternizing agents for producing quaternary ammonium salts include alkyl halides such as methyl chloride and methyl iodide; diethyl sulfate; and di-n-propyl sulfate.

Specific examples of preferred monomers (a₁) include quaternary ammonium salts, obtained through quaternization by use of a quaternizing agent, and acid-neutralized products of dimethylaminoethyl (meth)acrylate, diethylaminoethyl (meth)acrylate, dimethylaminopropyl(meth)acrylamide, or diethylaminopropyl(meth)acrylamide, and dimethyldiallylammonium chloride.

Examples of the monomers (a₂) include monomers represented by formula (2):

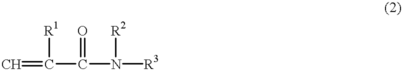

(wherein R¹ is the same as defined above; R² and R³ are the same as defined above or R² and R³ are linked to each other to represent —(CH₂)ₙ— (n is an integer of 3-6) or —(CH₂)₂O—(CH₂)₂—, forming a ring structure along with the adjacent nitrogen atom) or represented by formula (3):

[chemical structure]

(wherein R¹ is the same as defined above and E represents —(CH₂)ₘ— (m is an integer of 2-5)).

Specific examples of preferred monomers (a₂) include N-methyl(meth)acrylamide, N,N-dimethyl(meth)acrylamide, N,N-diethyl(meth)acrylamide, N-n-propyl(meth)acrylamide, N-t-butylacrylamide, N-(meth)acryloylmorpholine, N-vinylpiperidone, and N-vinylpyrrolidone. Of these, N,N-di-substituted-acrylamides are preferred in view of feeling for use of the composition. Moreover, such acrylamides; e.g., N,N-dimethyl(meth)acrylamide and N,N-diethyl(meth)acrylamide are particularly preferred.

The mol ratio of monomer (a₁) to monomer (a₂) ((a₁)/(a₂)) is preferably 2/98-98/2, more preferably 3/97-60/40. When the above mol ratio is low, thixotropic properties are readily manifested, whereas when the mol ratio is high, viscosity of the composition during application of a low shear stress is readily maintained. The mol ratio preferably falls within the above ranges so as to obtain the above two properties in a balanced manner. The ratio of the total amount of monomer (a₁) and monomer (a₂) to the total amount of all monomers is preferably 90% or more, more preferably 95% or more, particularly preferably 97% or more.

Examples of monomers (a₃) include (meth)acrylic acid esters of polyhydric alcohol or unsaturated alcohol; acrylamide; divinyl compounds; and polyallyl compounds. Of these, ethylene glycol di(meth)acrylate, polyethylene glycol di(meth)acrylate, pentaerythritol tetra(meth)acrylate, allyl-etherified pentaerythritol, vinyl (meth)acrylate, allyl (meth)acrylate, and similar compounds are particularly preferred.

The ratio of monomer (a₃) to the entirety of the monomers is preferably 0.002-5%, particularly preferably 0.002-3%, further preferably 0.002-1%, still further preferably 0.002-0.5%. When the ratio falls within the aforementioned ranges, the hydrogen formed from copolymer (A) provides soft and smooth touch.

Regarding copolymer (A), copolymers formed from at least one of the aforementioned preferred monomers of each monomer component are preferred. Particularly, copolymers formed from structural monomers; i.e., dimethylaminoethyl (meth)acrylate, N,N-dimethyl(meth)acrylamide, and polyethylene glycol di(meth)acrylate are preferred in view of deodorant effect.

In addition to essential structural units; i.e., at least one of the aforementioned vinyl monomers of each of three components, copolymer (A) may further contain, as a structural unit, a (meth)acryloyl-group-containing vinyl monomer represented by formula (4):

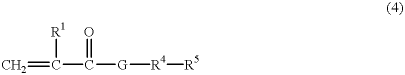

(wherein R¹ represents a hydrogen atom or a methyl group; G represents an oxygen atom or an —NH— group; R⁴ represents a C1-C17 linear or branched alkylene group or a group represented by formula (5):

(C_{q}H_{2q}O)ₚ (5)

(wherein q is an integer of 1-4 and p is an integer of 1-25), R⁵ represents a hydrogen atom or a methyl group).

In addition, copolymer (A) may further contain, as a structural unit, other vinyl monomers such as anionic-group-containing monomers; e.g., acrylic acid and methacrylic acid, and betaines such as N-(3-sulfopropyl)-N-acryloyloxyethyl-N,N-dimethylammonium betaine and N-carboxymethyl-N-methacryloyloxyethyl-N,N-dimethylammonium betaine.

Copolymer (A) can be produced through a routine method such as radical polymerization. Methods such as aqueous solution polymerization, reverse-phase suspension polymerization, and precipitation polymerization may be used.

Copolymer (A) can transform an aqueous medium into gel, and thus is a preferred component. Particularly, copolymer (A) is suitable for gelling an aqueous ethanol medium when used in combination with water.

Examples of the cationic cross-linked copolymer include the copolymer disclosed in Japanese Patent Application Laid-Open (kokai) No. 5-140531.

Poly((meth)acrylic acid) is a polymer of (meth)acrylic acid (monomer (b₁)), and a (meth)acrylic acid-alkyl (meth)acrylate (C1-C30) copolymer is a copolymer containing, as essential monomers, at least one species of monomer (b₁) and at least one species of alkyl (meth)acrylate (monomer (b₂)). Alkyl (meth)acrylate contains a C1-C30 alkyl group, preferably a C10-C30 alkyl group.

Cross-linked poly((meth)acrylic acid) contains, as an essential monomer, at least one species of monomer (b₁) and monomer (a₃), and a cross-linked (meth)acrylic acid-alkyl (meth)acrylate copolymer contains, as essential monomers, at least one species of monomer (b₁), at least one species of monomer (b₂), and at least one sepcies of monomer (a₃). In this case, the amount of monomer (a₃) is 0.00001-5%, preferably 0.00002-3%, more preferably 0.00005-1%, on the basis of the entirety of the monomers.

Examples of particularly preferred thickeners include copolymer (A) and (meth)acrylic acid-alkyl (meth)acrylate copolymers.

Two or more thickeners may be used, and the amount of a thickener incorporated into the deodorant composition may be determined so as to adjust the viscosity of the composition to 1,000-30,000 mPa·s (at 25° C.). Usually, the amount of a thickener contained in the composition is 0.01-10%, preferably 0.05-5%, on the basis of the entirety of the composition.

The viscosity of the deodorant composition of the present invention may be regulated by adding a solvent thereto. Particularly, a volatile solvent is preferably used.

Examples of volatile solvents include water, C1-C4 lower alcohols, cyclic dialkyl silicone having a polymerization degree of 4-5 (particularly, cyclic dimethyl silicone (e.g., Silicone SH-344: product of Dow Corning Toray)), chain dialkyl silicone having a polymerization degree of 2-5 (particularly, chain dimethyl silicone (e.g., Silicone KF-96L (2cs): product of Shin-Etsu Chemical Co., Ltd.)), and light paraffin (e.g., Nisseki Isosol 300: product of Nippon Petrochemicals Co., Ltd.). Of these, water or a water-soluble solvent are preferable. Particularly, a solution mixture of water and a lower alcohol is preferable. The weight ratio of water to a lower alcohol is 1/99-50/50, preferably 10/90-50/50, more preferably 20/80-45/55.

When the ratio of water to a lower alcohol falls within the above range, the deodorant composition is quickly dried, and other effects are obtained. For example, the composition easily imparts dry feeling to the skin when the composition is used in combination with powder. Preferred examples of lower alcohols include ethanol and isopropanol. Of these, ethanol is more preferable. The amount of a volatile solvent contained in the deodorant composition is usually 40-99%, preferably 60-99%, more preferably 80-99%.

The deodorant composition may contain, instead of water or a C1-C4 lower alcohol, a solvent having a solubility of 5 in water at 20° C. Examples of such solvents include polyalcohols and glycol ether.

When the deodorant composition of the present invention is used in combination with water-insoluble powder, the deodorant composition can impart more pleasant dry feeling to the skin.

Examples of the powder include organic powder such as polymer micropowder obtained through dispersion polymerization of a vinyl monomer in a solvent by use of, as a dispersant, a polysiloxane compound having a radical-polymerizable group on its one end (hereinafter the micropowder will be referred to as “polymer beads S”), silicone resin (e.g., KMP-590 (product of Shin-Etsu Chemical), Tospearl 145, Tospearl 2000B (products of Toshiba Silicone Co., Ltd.), Trefil (product of Toray)), nylon resin (e.g., SP-500 (product of Toray), polystyrene resin (e.g., Finepearl (product of Sumitomo Chemical Co., Ltd.), Techpolymer SB (product of Sekisui Plastics CO., Ltd.), Finepowder SGP (product of Soken Chemical Engineering Co., Ltd.)), polyethylene resin (e.g., FLO-BEADS (product of Sumitomo Seika Chemicals Co., Ltd.), polymethyl methacrylate resin (e.g., Microsphere M (product of Matsumoto Yushi-Seiyaku Co., Ltd.), Techpolymer MB (product of Sekisui Plastics CO., Ltd.), Finepowder MP (product of Soken Chemical Engineering Co., Ltd.)), divinylbenzene resin, synthetic silica beads, polyurethane resin, benzoguanamine resin, melamine resin, phenol resin, and fluorine-containing resin; and inorganic powder such as talc, sericite, mica, kaolin, red ion oxide, clay, bentonite, silicic acid, and mica.

The powder may assume any form among spherical, columnar, plate-like, and needle-like. Of these, the powder preferably assumes a spherical form, from the viewpoint of tribology. The true sphericity of the powder is not particularly limited, but the powder preferably has high sphericity. This is because, when the sphericity of the powder increases, the dynamic friction resistance of the deodorant composition decreases, and thus the composition imparts enhanced smooth feeling to the skin.

The mean particle size of the powder (as obtained through laser diffraction/scattering) is 0.05-50 μm, preferably 0.5-50 μm.

The powder may be subjected to treatment for imparting hydrophobicity, such as silicone treatment, fluorine treatment, metallic soap treatment, or fatty acid treatment, by means of a customary method.

The deodorant composition may contain two or more powders. In order to obtain a satisfactory effect of the powder and to prevent the skin from becoming white after application of the composition, the amount of the powder contained in the entire composition is 0.1-40%, preferably 1-20%, more preferably 2-8%.

In the present invention, when powder is incorporated into the composition, in accordance with other ingredients, there is obtained a deodorant composition in which the powder uniformly disperses. Alternatively, there is obtained a two-layer-separation-type deodorant composition in which the powder precipitates when the composition is allowed to stand. When such a two-layer-separation-type deodorant composition is used, the powder may be uniformly dispersed by application of vibration to the composition. When a cationic cross-linked copolymer having a quaternary ammonium salt, serving as a thickener, is incorporated into the two-layer-separation-type deodorant composition, dispersiblility of the powder is greatly enhanced.

When an oily ingredient is incorporated into the deodorant composition of the present invention, the relative dynamic friction resistance may be regulated, and the composition imparts desired dry feeling to the skin. In addition, when powder is incorporated into the composition, the composition exerts more excellent effects.

Examples of oily ingredients include silicon oils such as dimethylpolysiloxane, methylpolysiloxane dimethylcyclopolysiloxane, and methylhydrogenpolysiloxane; hydrocarbons such as solid or liquid paraffin, crystal oil, ceresine, ozocerite, and montan wax; vegetable or animal fats and oils and waxes such as olive oil, earth wax, carnauba wax, lanolin, and spermaceti; fatty acids and esters thereof such as stearic acid, palmitic acid, oleic acid, glycerin monostearate, glycerin distearate, glycerin monooleate, isopropyl myristate, isopropyl stearate, butyl stearate, and neopentyl glycol dicaprate; and higher alcohols such as cetyl alcohol, stearyl alcohol, palmityl alcohol, and hexyldodecyl alcohol. Of these, isopropyl myristate, neopentyl glycol dicaprate, or dimethylpolysiloxane is preferable.

When an oily ingredient is incorporated into the deodorant composition, the amount of the oily ingredient is 0.05-10%, preferably 0.1-5%.

Examples of preferred combinations of ingredients contained in the deodorant composition of the present invention include a combination of each preferable ingredient. Preferably, the deodorant composition contains a solvent mixture of water and ethanol, serving as a solvent; copolymer (A) (particularly preferably dimethylaminoethyl (meth)acrylate/N,N-dimethyl (meth)acrylamide/polyethylene glycol di(meth)acrylate, or (meth)acrylic acid-alkyl (meth)acrylate (C1-C30) copolymer), serving as a thickener; and a nonionic disinfectant (particularly, 3,4,4-trichlorocarbanilide, triclosan, or isopropylmethylphenol). When powder is incorporated into the composition, spherical powder; particularly, silicone resin spherical powder, is preferably used.

When the aforementioned ingredients are incorporated in an appropriate combination, the resultant deodorant composition has desired physical properties. For example, when a large amount of a highly volatile solvent is incorporated into the composition, the drying rate of the composition increases. The drying rate is also affected by other ingredients. For example, when a large amount of powder is incorporated into the composition, the drying rate of the composition tends to increase due to an increase in effective surface area. In contrast, when a large amount of a thickener is incorporated into the composition, if the conditions in relation to other ingredients remain unchanged, the drying rate of the composition tends to decrease. Usually, the relative dynamic friction resistance is reduced by incorporating into the composition a large amount of powder (a portion or all of the powder may be replaced by an oily ingredient). The relative dynamic friction resistance may also be affected by other ingredients. For example, the relative dynamic friction resistance may be affected by the type or amount of a thickener incorporated into the composition. The viscosity of the composition increases when a large amount of a thickener is incorporated into the composition. The viscosity may also be affected by selection of other ingredients. For example, when a good solvent with respect to a thickener is used as a volatile solvent, the viscosity of the composition may increase. By regulating the aforementioned factors, a variety of formulations are available within the scope of the present invention; for example, a formulation satisfying high drying rate for realization of dry feeling, or a formulation satisfying high viscosity for realization of easy handling.

The deodorant composition of the present invention may be mixed with other optional ingredients, to thereby provide a variety of product forms, such as transparent liquid, emulsion, gel, and lotion. In consideration of easy application to the skin and deodorant effects, the product form is preferably gel, lotion, or emulsion.

The deodorant composition of the present invention is preferably used by applying the necessary amount thereof to skin in need of deodorant.

### EXAMPLES

### Example Production of Copolymer (A)

### Production Example 1

A reactor was charged with an aqueous monomer solution containing N,N-dimethylaminoethyl methacrylate diethylsulfate (MOEDES: 80% aqueous solution, product of Nitto Kagaku Kogyo; 50.6 g), N,N-dimethylacrylamide (51.55 g), polyoxyethylene (14) glycol dimethacrylate (NK-14G: product of Shin-Nakamura Kagaku; 0.096 g), and ion exchange water (350 g). Note that the monomer solution had been previously purged with nitrogen. N₂ was further blown into the mixture for a further 20 minutes, and while the reaction system is purged with nitrogen, the temperature was raised to 55° C. Subsequently, a polymerization initiator (2,2′-azobis(2-amidinopropane) dihydrochloride; 0.19 g) was added. Polymerization took place when 30 minutes to one hour had elapsed, making the entire mixture a soft gel. Stirring was continued, and when four hours had elapsed after addition of the polymerization initiator, polymerization was stopped. Pellet was removed from the reactor, washed for 10 minutes in 5-L ethanol, and dried. Thereafter, the dried matter was pulverized in a coffee mill or jet mil. The pulverized particles were classified with HIVOLTER, yielding No. 1 copolymer (A).

### Production Example 2

The procedure of Production Example 1 was repeated using, N,N-dimethylaminoethyl methacrylate diethylsulfate (MOEDES, product of Nitto Kagaku Kogyo; 23.85 g), N,N-dimethylacrylamide (71.37 g), polyoxyethyleneglycol dimethacrylate (NK-9G: product of Shin-Nakamura Kagaku; 0.0429 g), ion exchange water (350 g), and a polymerization initiator ((2,2′-azobis(2-amidinopropane) dihydrochloride; 0.22), yielding No. 2 copolymer (A).

Table 1 shows the identity and proportions of the monomers constituting the cationic cross-linked copolymers which were similarly prepared and are used in the following Working Examples.

TABLE 1

| | Cationic- | Amido- | | |
|---|---|---|---|---|
| | group- | group- | Cross- | |
| | containing | containing | linkable | |
| | vinyl | vinyl | vinyl | |
| Copolymer | monomer | monomer | monomer | Proportions |
| (A) No. | (a₁) | (a₂) | (a₃) | (by mol) |
| 1 | MOEDES | DMAAm | NK-14G | 30/70/0.02 |
| 2 | MOEDES | DMAAm | NK-9G | 10/90/0.01 |
| 3 | MOEDSE | DMAAm | NK-14G | 20/80/0.01 |
| 4 | MOEDES | DMAAm | NK-9G | 20/80/0.02 |
| 5 | DMAPAA-DES | NMeAAm | NK-9G | 20/80/0.02 |

MOEDES: N,N-dimethylaminoethyl methacrylate diethylsulfate

DMAPAA-DES: N,N-dimethylaminopropylacrylamide diethylsulfate (a₂)

DMAAm: N,N-dimethylacrylamide

NMeAAm: N-methylacrylamide (a₃)

NK-9G: Polyoxyethylene (9) glycol dimethacrylate

NK-14G: Polyoxyethylene (14) glycol dimethacrylate

### Working Example 1

The deodorant compositions shown in Table 2 were prepared, and evaluated in terms of non-sticky feel and pleasant dry feel on the basis of the following standards. The results are also shown in Table 2.

Non-sticky Feel and Pleasant Dry Feel

Ten panelists applied respective compositions to their forearms for evaluation of pleasant dry feel and non-sticky feel in accordance with the following criteria.

Evaluation Score

Pleasant Dry Feel:

4: Instantaneous feeling of pleasant dry feel

3: Somewhat less instantaneous feeling of pleasant dry feel

2: Feeling of pleasant dry feel being not acknowledged very much

1: Feeling of pleasant dry feel being not acknowledged at all

Non-sticky Feel:

4: Excellent level of non-sticky feel

3: Fair level of non-sticky feel

2: Somewhat poor level of non-sticky feel

1: Poor level of non-sticky feel

Averaged evaluation score:

3.5-4.0 ⊚

2.5-3.4 ◯

1.5-2.4 Δ

1.0-1.4 X

TABLE 2

| | | | (%) | |
|---|---|---|---|---|
| | Invention | | Comparative | |
| | products | | products | |
| | 1 | 2 | 1 | 2 |
| Deodorant compositions | | | | |
| (a) Isopropylmethylphenol | 0.1 | 0.1 | 0.1 | 0.1 |
| (b) Silicone powder¹⁾ | 6.0 | 5.0 | — | |
| (c) Ethanol | 70.0 | 80.0 | 70.0 | 25 |
| (Meth)acrylic acid/alkyl | 0.3 | — | 0.3 | 0.3 |
| (meth)acrylate (C10-C30) | | | | |
| copolymer²⁾ | | | | |
| Triethanolamine | 0.075 | — | 0.075 | 0.075 |
| No.1 Copolymer (A) | — | 0.3 | — | — |
| Purified water | to 100 | to 100 | to 100 | to 100 |
| Isopropyl myristic acid | — | 0.5 | — | — |
| Viscosity mPa · s (B-type | 6500 | 2500 | 5300 | 16000 |
| viscometer, rotor 3, 25° C.) | | | | |
| Drying speed(sec) | 18 | 16 | 22 | 45 |
| Dynamic friction resistance | 23 | 26 | 95 | 38 |
| (%) | | | | |
| Smooth feel | ⊚ | ⊚ | X | ◯ |
| Perception of pleasant dry | ⊚ | ⊚ | Δ | Δ |
| feel | | | | |

Note)
¹⁾KMP-590 (product of Shin'etsu Chemical Co., Ltd.); mean particle size: 2 micrometers (laser diffraction/diffusion size-distribution measurement apparatus model LA-910 (manufactured by Horiba Seisakusho)
²⁾Carbopol ETD 2020 (product of B. F. Goodrich)

The products according to the present invention dried quickly, provided smooth feel and pleasant dry feel, and thus were found to be excellent deodorant compositions.

### Working Example 2

The deodorant compositions shown in Table 3 were prepared. 15 panelists with body odor used the respective compositions by applying them to their underarms (0.5 g each). Eight hours following the application, the panelists evaluated deodorant effect of the compositions in accordance with the following criteria. Simultaneously, the panelists evaluated pleasant dry touch to the skin feeling by means of sensory evaluation. The re-dispersibility of the powder was evaluated through visual observation. Briefly, a deodorant composition (70 g) was placed in each of ten 100-mL glass containers, and allowed to stand for one month in a thermostat chamber. Thereafter, the containers were shaken and the state of dispersion was visually checked.

Evaluation Score for Deodorant Effect:

4: Excellent deodorant effect

3: Good deodorant effect

2: Moderate deodorant effect

1: Poor deodorant effect

Evaluation Score for Pleasant Dry Touch to the Skin:

4: Excellent

3: Good

2: Moderate

1: Poor

Evaluation Score for Re-Dispersibility of Powder:

4: Excellent

3: Good

2: Moderate

1: Poor

Table 3 shows the evaluation results summarized by use of averaged evaluation ratings obtained in a manner similar to that described in relation to Working Example 1.

TABLE 3

| | | (%) |
|---|---|---|
| | Invention | Comparative |
| | products | products |
| | 3 | 3 |
| No. 4 Copolymer (A) | 0.3 | |
| Hydroxyethylcellulose¹⁾ | | 0.3 |
| Triclosan | 0.1 | 0.1 |
| Silicone powder²⁾ | 3 | 3 |
| Ethanol | 50 | 20 |
| Water | to 100 | to 100 |
| Deodorant effect | ⊚ | Δ |
| Pleasant dry touch to the | ⊚ | X |
| skin | | |
| Re-dispersibility of | ⊚ | ◯ |
| powder | | |

Note)
¹⁾HEC SE850 (product of Dicel Kagaku Kogyo)
²⁾KMP-590 (product of Shin'etsu Chemical Co., Ltd.)

The deodorant compositions of Invention products 1 and 2 provided excellent deodorant effect and pleasant dry touch to the skin. Also, the invention product 2 exhibited excellent re-dispersibility of powder.

### Working Example 3

The deodorant compositions shown in Table 4 were prepared. All the compositions provided excellent deodorant effect, preventing sweat odor and foot odor, and also exhibited pleasant dry touch to the skin.

TABLE 4

| | (%) | | |
|---|---|---|---|
| | Invention products | | |
| | 4 | 5 | 6 |
| No. 2 Copolymer (A) | 0.4 | | |
| No. 3 Copolymer (A) | | 0.3 | |
| No. 5 Copolymer (A) | | | 0.3 |
| Isopropyl methylphenol | 0.1 | 0.1 | 0.1 |
| Trichlorocarbanilide | | | 0.2 |
| Silicone powder¹⁾ | 3 | | 6 |
| Silicone-treated nylon powder²⁾ | 3 | | |
| Polystyrene powder³⁾ | | 3 | |
| Ethanol | 75 | 90 | 80 |
| Neopentyl glycol dicaprate | 0.5 | 1 | |
| α-Monoisostearyl glyceryl ether | | | 0.3 |
| Dimethylpolysiloxane (SH200C)⁴⁾ | | 0.2 | |
| Perfume/pH regulator/ | Suitable | Suitable | Suitable |
| Colorant/preservative | amount | amount | amount |
| Purified water | to 100 | to 100 | to 100 |

Note)
¹⁾KMP-590 (product of Shin'etsu Chemical Co., Ltd.)
²⁾Nylon SP-500S (product of Toray) 7 micrometers
³⁾Finepearl (Sumitomo Chemical Co., Ltd.) 10 micrometers
⁴⁾Product of Dow Corning Toray

### Working Example 4:

The following deodorant compositions were prepared. They dried quickly on the skin, and provided non-sticky feeling and pleasant dry touch to the skin. Also, they did not spill from the palms, thus proving excellent handling during use.

| | |
|---|---|
| Triclosan | 0.2% |
| Nylon powder¹⁾ | 20.0 |
| Ethanol | 40.0 |
| Cross-linked poly(meth)acrylic acid²⁾ | 0.3 |
| Triethanolamine | 0.3 |
| Purified water | balance (to make a total of 100) |

Note)
¹⁾Nylon powder SP-500 (product of Toray)
²⁾Carbopol 940 (product of B. F. Goodrich)

Industrial Applicability

The deodorant compositions according to the present invention exhibit excellent deodorant effects against body odor, and their effects last for long periods of time. They dry quickly on the skin, impart soft feel to the skin, and provide pleasant dry touch to the skin. Also, they do not spill from the palms and thus can be handled conveniently.

1. A deodorant composition containing a disinfectant or an antiperspirant, and a thickener, which composition dries within a drying time of 30 seconds, exhibits a relative dynamic friction resistance of 50% or less, and has a viscosity of 1,000-30,000 mpa·s.

2. The deodorant composition according to claim 1, wherein the thickener is a cationic cross-linked copolymer or a (meth)acrylic polymer.

3. The deodorant composition according to claim 2, wherein the cationic cross-linked copolymer is obtained through radical polymerization of monomers comprising at least one cationic-group-containing vinyl monomer, at least one amido-group-containing vinyl monomer, and at least one cross-linkable vinyl monomer containing two or more vinyl groups in the molecule.

4. The deodorant composition according to claim 3, wherein the amido-group-containing vinyl monomer is represented by formula (2): 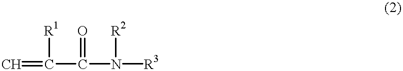
(wherein R¹ is a hydrogen atom or a methyl group; each of R² and R³, which are same or different from each other, represent a hydrogen atom or a C1-C4 linear or branched alkyl or alkenyl group, or R² and R³ are linked to each other to represent —(CH₂)ₙ— (n is an integer of 3-6) or —(CH₂)₂O—(CH₂)₂—, to thereby form a ring structure together with the adjacent nitrogen atom) or represented by formula (3): [chemical structure]
(wherein R¹ is the same as defined above and E represents —(CH₂)ₘ— (m is an integer of 2-5)).

5. The deodorant composition according to claim 2, wherein the (meth)acrylic polymer is a copolymer of (meth)acrylic acid and C1-C30 alkyl (meth)acrylate.

6. The deodorant composition according to claim 1, further containing a volatile solvent.

7. The deodorant composition according to claim 6, wherein the volatile solvent is water-soluble.

8. The deodorant composition according to claim 6, wherein the volatile solvent is a mixture of water and a lower alcohol at a weight ratio of water to the lower alcohol of 1/99-50/50.

9. The deodorant composition according to claim 1, further containing a powder.

10. A deodorant method comprising applying to the skin a composition containing a disinfectant or an antiperspirant, and a thickener, which composition dries within a drying time of 30 seconds, exhibits a relative dynamic friction resistance of 50% or less, and has a viscosity of 1,000-30,000 mPa·s.
